(19) United States
(12) Patent Application Publication
(10) Pub. No.: US 2023/0147530 A1
(43) Pub. Date: May 11, 2023

(54) Antiviral Composition for Oral Care

(71) Applicants: Advance NanoTek Limited, Rocklea (AU); Astivita Limited, Rocklea (AU)

(72) Inventors: Dalia Mizikovsky, Rocklea (AU); Joseph Mizikovsky, Rocklea (AU)

(21) Appl. No.: 17/906,584

(22) PCT Filed: Mar. 17, 2021
(86) PCT No.: PCT/AU2021/050238

§ 371 Date: Sep. 16, 2022

(30) Foreign Application Priority Data
Mar. 17, 2020 (AU) 2020900820

(51) Int. Cl. A61K 33/30; A61Q 11/00; A61K 8/27; A61K 9/107; A61K 9/00; A61P 31/14; A61K 47/26

(52) U.S. Cl. CPC A61K 33/30; A61Q 11/00; A61K 8/27; A61K 9/107; A61K 9/006; A61P 31/14; A61K 47/26

(57) ABSTRACT

Viral infection of the oral mucosa (the lining of the oral cavity) is a common feature of many respiratory viruses that is central to pathogenesis and spread. Despite this, few anti-viral oral hygiene products exist. The present invention is a composition of a zinc salt and hinokitiol to be used as an anti-viral agent in oral care products. The composition is extremely low risk for adverse effects and irritation, and as such can be used continuously and frequently as both a preventative measure and a treatment. Oral care products in which this composition may be used include, but are not limited to toothpaste, mouth rinse and gargling solution. The composition inhibits viral replication in the oral cavity through interacting with the replication machinery of the viruses.

### TECHNICAL FIELD

The present invention relates to the field of anti-viral products for the oral cavity. Specifically, it relates to an anti-viral composition which can be widely used in oral care products for the prevention and treatment of viral infections.

### BACKGROUND ART

Viral infections of the oral mucosa (lining of the oral cavity) are commonly encountered in general practice. In addition to viruses which specifically infect the oral mucosa, such as the herpes simplex virus, many common respiratory viruses, such as the influenza virus, also infect the oral mucosa during their pathogenesis. The replication and release of respiratory viruses in the oral cavity contributes both to their pathogenesis and their contagion.

Despite the importance of the oral cavity in the pathogenesis and contagion of respiratory viruses, there are very few anti-viral products for the treatment of the oral mucosa available. Existing anti-viral products are only suitable to target localized infections, such as cold sores caused by the herpes simplex virus. Moreover, the anti-viral products are often specific to the condition—i.e. will only function for the herpes simplex virus. Finally, they may only be used after an infection has developed.

Therefore, a need exists in the field for a composition to be widely used in oral care products with broad anti-viral properties. There further exists a need for a composition that could be utilized preventatively, in addition to in response to an infection. In this niche, the composition must be safe for frequent and continuous use, with minimal risks and side-effects.

It will be clearly understood that, if a prior art publication is referred to herein, this reference does not constitute an admission that the publication forms part of the common general knowledge in the art in Australia or in any other country.

### SUMMARY OF INVENTION

In one aspect, the present invention provides a composition for use in oral care comprising at least one water soluble zinc salt and a zinc ionophore selected from one or more of hinokitiol, quecertin, epigallocatechin, and zincophorin or a salt thereof.

In one embodiment, the composition contains at least one zinc salt, or two or more zinc salts.

In one embodiment, the at least one water soluble zinc salt may be selected from one or more of zinc chloride, zinc myristate, zinc citrate, zinc nitrate, or zinc sulfate, zinc chlorate, zinc phosphate, zinc molybdate, zinc chromate, zinc bromide and zinc iodide. Other zinc salts that are non-toxic may also be used. The zinc salts may be inorganic salts or organic salts.

In one embodiment, the composition comprises a therapeutically effective amount of at least one water soluble zinc salt and hinokitiol, quecertin, epigallocatechin, and zincophorin.

In one embodiment, the zinc ionophore comprises hinokitiol or a salt thereof.

The composition of the present invention shows anti-viral properties. The composition of the present invention is suitable for use in oral care products, such as, but not limited to, mouth rinse, toothpaste, gargling solutions and mouth wash. In other embodiments, the composition may comprise a throat lozenge or a nasal spray or a topical oral mucosa treatment composition or a topical nasal mucosa treatment or an inhaler or a puffer or a treatment for the oro-pharynx. Products containing this composition may be used continuously and frequently, for the prevention and treatment of viral infections.

The composition takes advantage of the combinatorial effect of zinc ions, released from a zinc salt when dissolved, and hinokitiol, which promotes the uptake of zinc ions into cells.

Hinokitiol is a presently preferred zinc ionophore for use in the present invention. Any therapeutically acceptable salt of hinokitiol or any pharmaceutically acceptable salt of hinokitiol may be used in the present invention. However, preferred embodiments may use hinokitiol. The IUPAC name of hinokitiol is 2-Hydroxy-6-propan-2-ylcyclohepta-2,4,6-trien-1-one.

In one embodiment, the composition comprises zinc present in an amount of 0.1 wt %, calculated as zinc chloride equivalent, and hinokitiol is present in an amount of from 0.01 to 0.2 wt %. In this embodiment, the oral care composition may be used as a prophylactic to prevent a viral infection or to minimise a likelihood of viral infection. In this embodiment, the oral care composition may comprise a mouth wash or a gargling liquid, a throat lozenge, a topical oral mucosa treatment composition or a topical nasal mucosa treatment or an inhaler or a puffer or a treatment for the oro-pharynx.

In another embodiment, the composition comprises zinc present in an amount of 0.1 wt %, calculated as zinc chloride equivalent, and hinokitiol is present in an amount of from 0.3 to 1.0 wt %. In this embodiment, the oral care composition may be used as a treatment for treating a viral infection. In this embodiment, the oral care composition may comprise a mouth wash or a gargling liquid, a throat lozenge, a topical oral mucosa treatment composition or a topical nasal mucosa treatment or an inhaler or a puffer or a treatment for the oro-pharynx.

In another embodiment, the composition comprises a toothpaste comprising zinc present in an amount of from 0.75 to 1.0 wt %, calculated as zinc chloride equivalent, and hionkitiol present in an amount of from 0.01 to 1.0 wt %.

In embodiments where the zinc ionophore is one or more of quecertin, epigallocatechin, and zincophorin, these components may be present in the amounts specificed for hinokitiol in the three preceding paragraphs.

Throughout this specification, all weight percentages are given as a weight percentage of the overall composition. For example, if the composition is stated to contain 0.1 wt % hinokitiol, 100 g of the composition contains 0.1 g hinokitiol.

In one embodiment, the composition comprises an aqueous composition. In another embodiment, the composition comprises an emulsion or a dispersion. In one embodiment, the composition comprises an oil-in-water emulsion.

In one embodiment, the composition comprises one or more sweeteners. The one or more sweeteners may be present in an amount of up to 75 wt %, or up to 50 wt %, or up to 25 wt %, or up to 20 wt %, or up to 15 wt %. The sweetener may comprise one or more of sorbitol, glycerin, inositol, saccharine or sodium saccharine. In one embodiment, the composition comprises sorbitol in an amount of from 5-10 wt %. In one embodiment, the composition comprises xylitol in an amount of from 1-5 wt %. In one embodiment, the composition comprises glycerin in an amount of from 1-5 wt %. A toothpaste will typically have a higher amount of sweetener than a mouthwash, with a toothpaste containing up to 70 wt % sweetener.

The composition may also comprise one or more flavour components or flavour masking agents. These will generally be present in an amount of less than 5 wt %. The composition may also comprise one or more preservatives or one or more biocides.

The composition may contain one or more gelling agents or gels, particularly when the composition comprises a toothpaste.

In one embodiment, the composition comprises an oil-in-water emulsion or a dispersion comprising an oil phase dispersed in an aqueous phase, the oil phase comprising less than 10 wt %, or less than 7.2 wt % of the overall composition. In this embodiment the composition may also comprise a surfactant. The surfactant may be present in an amount of up to 5 wt %, or up to 2 wt %, or from 0.1 to 1 wt %.

In embodiments where the composition comprises a toothpaste, the toothpaste may also comprise tooth care components and enamel care components, such as fluoride compounds used in known toothpastes, flavour components, and preservatives. Effectively, the toothpaste may comprise a known toothpaste composition that also includes one or more soluble zinc salts and hinokitiol or a salt thereof.

Other ingredients may also be added to the composition, such as plant extracts with natural anti-inflammatory, antiseptic or antibacterial action. Mint and eucalyptus extracts are examples of this. Breath fresheners, or ingredients that mask or treat bad breath, may also be added.

In a second aspect, the present invention provides use of at least one water soluble zinc salt and a zinc ionophore selected from one or more of hinokitiol, quecertin, epigallocatechin, and zincophorin or a salt or salts thereof in the preparation of a medicament composition for treating viral infections. The medicament composition may comprise an oral care composition, as described above.

In a third aspect, the present invention provides a method for treating a viral infection by treating a patient with a composition as described reference to the first aspect of the present invention. The composition may comprise an oral care composition and the patient may use the oral care composition by taking it into the mouth. In one embodiment, the oral care composition may be gargled, or used as a toothpaste or a mouthwash. The composition could be subsequently spat out or it may not be spat out. In another embodiment, the composition may be used as an oral spray and not spat out.

In a fourth aspect, the present invention provides use of a composition as described reference to the first aspect of the present invention for treating or preventing a viral infection or lowering risk of viral infection or reducing viral load in a patient. The composition may comprise an oral care composition and the patient may use the oral care composition by taking it into the mouth. In one embodiment, the oral care composition may be gargled, or used as a toothpaste or a mouthwash and subsequently spat out or it may not be spat out. In another embodiment, the composition may be used as an oral spray and not spat out.

In one embodiment of the method, the composition comprises zinc present in an amount of 0.1 wt %, calculated as zinc chloride equivalent, and hionkitiol present in an amount of from 0.01 to 0.2 wt % and the method is a prophylactic method for avoiding or minimising likelihood of a viral infection occurring.

In another embodiment of the method, the composition comprises zinc present in an amount of 0.1 wt %, calculated as zinc chloride equivalent, and hionkitiol present in an amount of from 0.3 to 1.0 wt %, and the composition is used as a treatment for treating a viral infection of the oral mucosa or to reduce a viral load or to reduce symptoms and severity of viral infection or to prevent appearance of symptoms arising from viral infection and the oral care composition comprises a mouth wash or a gargling liquid, a throat lozenge, a topical oral mucosa treatment composition or a topical nasal mucosa treatment or an inhaler or a puffer or a treatment for the oro-pharynx.

In yet another embodiment of the method, the composition comprises a toothpaste comprising zinc present in an amount of from 0.75 to 1.0 wt %, calculated as zinc chloride equivalent, and hionkitiol present in an amount of from 0.01 to 1.0 wt % and the method comprises brushing the teeth, or inside the cheeks, or the tongue, of a patient for prophylactic or treatment of a viral infection of the oral mucosa.

The method of the third aspect and the fourth aspect may comprises a prophylactic treatment to prevent or at least minimise the likelihood of the patient being infected by a virus, or may comprise a method to reduce cellular viral load or to reduce symptoms and severity of viral infection or to prevent appearance of symptoms arising from viral infection.

In a fifth aspect, the present invention provides a composition as described herein for use in making an oral care composition for treating viral infections or for preventing or lowering risk of viral infection.

In a sixth aspect, the present invention provides a method for inhibiting viral replication in the oral cavity through interacting with the replication machinery of the viruses by applying an oral care composition as described with reference to the first aspect of the present invention to the oral mucosa.

The present invention may be used to treat a number of viruses, especially viruses that can infect the upper respiratory tract and the lower respiratory tract. Such viruses include rhinoviruses, corona viruses that cause colds, severe acute respiratory syndrome and COVID-19, and influenza viruses.

The composition of the present invention and methods of treatment of the present invention may be used by applying the composition to the mucosa of one or more of the nose, mouth, throat and lungs. Care should be taken when applying to the nose as zinc sprays/gels for the nose may cause loss of smell.

Any of the features described herein can be combined in any combination with any one or more of the other features described herein within the scope of the invention.

The reference to any prior art in this specification is not, and should not be taken as an acknowledgement or any form of suggestion that the prior art forms part of the common general knowledge.

### BRIEF DESCRIPTION OF DRAWINGS

Various embodiments of the invention will be described with reference to the following drawings, in which:

FIG. 1 depicts an illustration of the infection and replication process of an RNA virus, such as that of influenza. Zinc ions interact with the processing of viral polyproteins and the RNA dependent RNA polymerase (RdRp), inhibiting the replication of the viral genome and proteins. However, zinc ions are normally unable to pass through the membrane, due to possessing a charge. Hinokitiol promotes the uptake of zinc ions into cells through an uncharacterized mechanism, enabling maximal effect.

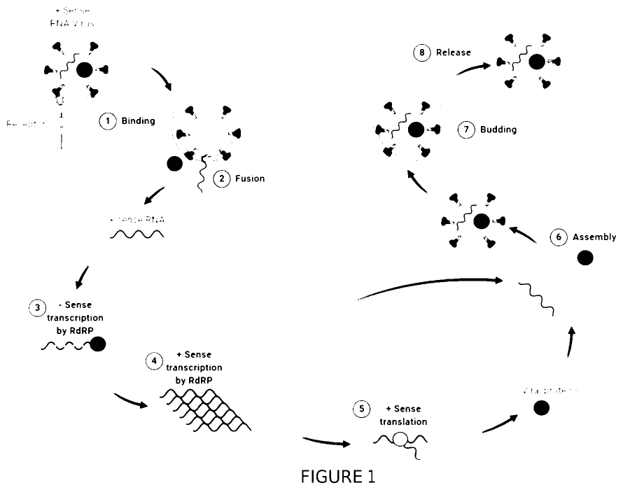

### DESCRIPTION OF EMBODIMENTS

The terminology used herein is for the purpose of describing particular embodiments only and is not intended to be limiting of the invention. As used herein, the term “and/or” includes any and all combinations of one or more of the associated listed items. As used herein, the singular forms “a”, “an”, and “the” are intended to include the plural forms as well as singular forms, unless the context clearly indicates otherwise. It will be further understood that the terms “comprises” and/or “comprising”, when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which the invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specifications and claims should be read with the understanding that such combinations are entirely within the scope of the invention and claims.

The present disclosure is to be considered as an exemplification of the invention, and is not intended to limit the invention to the specific embodiments illustrated by the figures or description below.

The present invention will now be described. The composition is comprised of hinokitiol and a zinc salt or zinc compounds, including but not limited to zinc chloride, zinc oxide, zinc nitrate, zinc chlorate, zinc sulfate, zinc phosphate, zinc molybdate and zinc chromate. Other zinc compounds or zinc salts may also be used. Two or more zinc compounds or zinc salts may be used. This composition may be used as an anti-viral active agent in oral care formulations. The proportions of hinokitiol and zinc are not limited, but are especially preferable in a ratio of hinokitiol to zinc of 1:3 to 10:1.

The anti-viral action of the zinc salt and hinokitiol composition occurs through multiple mechanisms of action. The present inventors have postulated that zinc ions derived from a zinc compound or a zinc salt inhibit the replication machinery of RNA viruses, and as such can help control illness and spread of the diseases. Hinokitiol strongly promotes the transport of zinc into cells, making this mode of action more effective. Common RNA viruses include the influenza, which affects at least 9 million people a year. Another RNA virus is the COVID-19, the coronavirus which has currently caused a pandemic. COVID-19 is very similar to the virus which caused severe acute respiratory syndrome (SARS). It was found that the SARS virus entered and replicated inside the oral mucosa, and then was released into saliva, facilitating its spread. A similar process has been predicted to occur with COVID-19.

Viral infection of the oral mucosa (the lining of the oral cavity) is a common feature of many respiratory viruses that is central to pathogenesis and spread. Despite this, few anti-viral oral hygiene products exist. The present invention in some embodiments is a composition of a zinc salt and hinokitiol to be used as an anti-viral agent in oral care products. The composition is extremely low risk for adverse effects and irritation, and as such can be used continuously and frequently as both a preventative measure and a treatment. Oral care products in which this composition may be used include, but are not limited to toothpaste, mouth rinse and gargling solution. The composition inhibits viral replication in the oral cavity through interacting with the replication machinery of the viruses.

### EXAMPLE 1

The following composition was prepared:

| Phase | Ingredient | Percentage |
|---|---|---|
| A | Aqua | 50-75 |
| A | Sorbitol | 5-10 |
| B | PEG-40 Hydrogenated Castor Oil | 1-5 |
| A | Xylitol | 1-5 |
| A | Glycerin | 1-5 |
| A | Poloxamer 407 | 0.1-1 |
| B | Eucalyptus Globulus Leaf Oil | <0.1 |
| B | Mentha Piperita Oil | 0.1-1 |
| A | Sodium Citrate | 0.1-1 |
| A | Inositol | <0.1 |
| A | Zinc Chloride | <0.1 |
| A | Sodium Saccharin | 0.1-1 |
| A | O-Cymen-5-OL | <0.1 |
| B | Hexetidine | <0.1 |
| A | Sodium Benzoate | <0.1 |
| B | Phenoxyethanol | 0.1-1 |
| A | Citric Acid | 0.1-1 |
| C | Hinokitiol | 0.1-1 |

This composition was prepared by combining the Phase A ingredients and heating to 60° C. Following this, the Phase B ingredients were combined and added to phase A under high shear to form a stable emulsion. Hinokitiol can be added either to phase A, B or to the complete emulsion.

The composition of Example 1 was tested on human HeLa cells infected with the feline coronavirus. Viral titre was measured after 5 minutes and after 1 hour. A log reduction of 3 represents a 99.9% reduction in virus titre, and a log reduction of 4 represents a 99.99% reduction. The following results were obtained:

| | Log-reduction in viral |
|---|---|
| Treatment, | titre (feline coronavirus) |
| Control, | 0 |
| 5 Minutes with | 3.25 |
| composition of example 1 | |
| 1 Hour with composition | 4.00 |
| of example 1 | |

The above results show a dramatic reduction in the viral load by use of the composition of example 1 in a short period of time.

In the present specification and claims (if any), the word ‘comprising’ and its derivatives including ‘comprises’ and ‘comprise’ include each of the stated integers but does not exclude the inclusion of one or more further integers.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims (if any) appropriately interpreted by those skilled in the art.

### CITATION LIST

### Patent Literature

Otsu, Yoshiro, Yaeno Arima, and Yoriko Nakai. 1997. “Antibacterial and Antifungal Activity Method, Therapeutic Method of Infectious Diseases and Preserving Method of Cosmetics.”—U.S. Pat. No. 5,696,169A
Fukui, Atsuko and Takuya Nakatsubo. 2019. “Antiviral Agent and Throat Candy, Gargle, and Mouthwash Using the Same.”—JP2019077617A
Dong, Liang, Steven Espinal, Patrick Wong, and Paul Magruder. 2007. “Antiviral Medication.”—ES2236012T3

### Non Patent Literature

Krenn, B. M. et al. 2009. “Antiviral Activity of the Zinc Ionophores Pyrithione and Hinokitiol against Picornavirus Infections.” Journal of Virology 83(1):58-64.
Te Velthuis, Aartjan J. W. et al. 2010. “Zn2+ Inhibits Coronavirus and Arterivirus RNA Polymerase Activity in Vitro and Zinc Ionophores Block the Replication of These Viruses in Cell Culture.” PLoS Pathogens 6(11):1-10.
Wang, Wei Kung et al. 2004. “Detection of SARS-Associated Coronavirus in Throat Wash and Saliva in Early Diagnosis.” Emerging Infectious Diseases 10(7):1213-19

Xu, Hao et al. 2020. “High Expression of ACE2 Receptor of 2019-NCoV on the Epithelial Cells of Oral Mucosa.” International Journal of Oral Science 12(1 PG-8):8. Retrieved (https://doi.org/10.1038/s41368-020-0074-xNS-).

1-36. (canceled)

37. A composition for use in oral care comprising at least one water soluble zinc salt and a zinc ionophore selected from one or more of hinokitiol, quercetin, epigallocatechin, and zincophorin or a salt thereof.

38. The composition as claimed in claim 37, wherein the composition contains at least one zinc salt, or two or more zinc salts; or
wherein the at least one water soluble zinc salt is selected from one or more of zinc chloride, zinc myristate, zinc citrate, zinc nitrate, or zinc sulfate, zinc chlorate, zinc phosphate, zinc molybdate, zinc chromate, zinc bromide and zinc iodide.

39. The composition as claimed in claim 37, wherein:
the composition comprises a therapeutically effective amount of at least one water soluble zinc salt and a therapeutically effective amount of one or more of hinokitiol, quercetin, epigallocatechin, and zincophorin;
the zinc ionophore comprises hinokitiol or a salt thereof; or
the composition comprises hinokitiol.

40. The composition as claimed in claim 37, wherein the composition comprises a mouth rinse, a toothpaste, a gargling liquid, a mouth wash, a throat lozenge, a nasal spray, a topical oral mucosa treatment composition, a topical nasal mucosa treatment, an inhaler or a puffer, or a composition for treating the oro-pharynx.

41. The composition as claimed in claim 37, wherein the composition comprises zinc present in an amount of 0.1 wt %, calculated as zinc chloride equivalent, and hinokitiol is present in an amount of from 0.01 to 0.2 wt %.

42. The composition as claimed in claim 37, wherein:
the composition comprises zinc present in an amount of 0.1 wt %, calculated as zinc chloride equivalent, and hinokitiol present in an amount of from 0.3 to 1.0 wt %, and the composition is used as a treatment for treating a viral infection and the oral care composition comprises a mouth wash or a gargling liquid, a throat lozenge, a topical oral mucosa treatment composition or a topical nasal mucosa treatment or an inhaler or a puffer or a treatment for the oro-pharynx; or
the composition comprises a toothpaste comprising zinc present in an amount of from 0.75 to 1.0 wt %, calculated as zinc chloride equivalent, and hinokitiol present in an amount of from 0.01 to 1.0 wt %.

43. The composition as claimed in claim 37, wherein the composition comprises an aqueous composition; or
wherein the composition comprises an emulsion or a dispersion, wherein the emulsion is an oil-in-water emulsion.

44. The composition as claimed in claim 37, wherein the composition comprises one or more sweeteners.

45. The composition as claimed in claim 44, wherein:
the one or more sweeteners are present in an amount of up to 75 wt %, or up to 50 wt %, or up to 25 wt %, or up to 20 wt %, or up to 15 wt %;
the one or more sweeteners comprise one or more of sorbitol, glycerin, inositol, saccharine or sodium saccharine; or
the composition comprises sorbitol in an amount of from 5-10 wt %, xylitol in an amount of from 1-5 wt %, and glycerin in an amount of from 1-5 wt %.

46. The composition as claimed in claim 37, further comprising: one or more flavor components or flavor masking agents, one or more preservatives or one or more biocides, or one or more gelling agents or gels.

47. The composition as claimed in claim 37, wherein the composition comprises an oil-in-water emulsion or a dispersion comprising an oil phase dispersed in an aqueous phase, the oil phase comprising less than 10 wt %, or less than 7.2 wt % of the overall composition.

48. The composition as claimed in claim 47, wherein the composition also comprises a surfactant.

49. The composition as claimed in claim 48, wherein the surfactant is present in an amount of up to 5 wt %, or up to 2 wt %, or from 0.1 to 1 wt %.

50. The composition as claimed in claim 37, further comprising plant extracts with natural anti-inflammatory, antiseptic or antibacterial action; or breath fresheners or ingredients that mask or treat bad breath.

51. A method for treating a viral infection or preventing a viral infection or lowering risk of viral infection or reducing viral load in a patient, by treating the patient with a composition as claimed in claim 37.

52. The method as claimed in claim 51, wherein the composition comprises an oral care composition and the patient uses the oral care composition by taking it into the mouth.

53. The method as claimed in claim 52, wherein the oral care composition is gargled, or used as a toothpaste or a mouthwash or as an oral spray.

54. The method as claimed in claim 51, wherein:
the composition comprises zinc present in an amount of 0.1 wt %, calculated as zinc chloride equivalent, and hinokitiol present in an amount of from 0.01 to 0.2 wt % and the method is a prophylactic method for avoiding or minimizing likelihood of a viral infection occurring;
the composition comprises zinc present in an amount of 0.1 wt %, calculated as zinc chloride equivalent, and hinokitiol present in an amount of from 0.3 to 1.0 wt %, and the composition is used as a treatment for treating a viral infection of the oral mucosa or to reduce a viral load or to reduce symptoms and severity of viral infection or to prevent appearance of symptoms arising from viral infection and the oral care composition comprises a mouth wash or a gargling liquid, a throat lozenge, a topical oral mucosa treatment composition or a topical nasal mucosa treatment or an inhaler or a puffer or a treatment for the oro-pharynx; or
the composition comprises a toothpaste comprising zinc present in an amount of from 0.75 to 1.0 wt %, calculated as zinc chloride equivalent, and hinokitiol present in an amount of from 0.01 to 1.0 wt % and the method comprises brushing the teeth, or inside the cheeks, or the tongue, of a patient for prophylactic or treatment of a viral infection of the oral mucosa.

55. The method as claimed in claim 51, wherein the method treats one or more selected from the following: viral infections caused by rhinoviruses; corona viruses that cause colds; severe acute respiratory syndrome and COVID-19; and influenza viruses.

56. A method for inhibiting viral replication in an oral cavity through interacting with replication machinery of one or more viruses by applying a composition as claimed in claim 37 to the oral mucosa.
